(19) United States
(12) Patent Application Publication
(10) Pub. No.: US 2008/0153077 A1
(43) Pub. Date: Jun. 26, 2008

(54) Substrates for immobilizing cells and tissues and methods of use thereof

(76) Inventor: David Henry, Morigny-Champigny (FR)

Correspondence Address: CORNING INCORPORATED, SP-TI-3-1, CORNING, NY, 14831, US

(21) Appl. No.: 11/811,099

(22) Filed: Jun. 8, 2007

(30) Foreign Application Priority Data
Jun. 12, 2006 (EP) 06300582.1

(51) Int. Cl. A01N 1/02; C12M 1/00; C12N 5/02; C12Q 1/02

(52) U.S. Cl. 435/1.1; 435/283.1; 435/398; 435/29; 435/375; 977/773

(57) ABSTRACT

Described herein are substrates for immobilizing cells and tissues and methods of use thereof.

### CROSS-REFERENCE TO RELATED APPLICATIONS

This application claims the benefit of European Application Serial No. 06300582.1 filed on Jun. 12, 2006 and entitled “Substrates for Immobilizing Cells and Tissues and Methods of Use Thereof” which is incorporated by reference herein.

### BACKGROUND

The ability to proliferate and differentiate cell growth in vivo has numerous applications. Cell growth in vivo occurs in the extracellular matrix (ECM), which is a three-dimensional environment. A two-dimensional surface such as a Petri dish surface for example, is not representative of cells growing “in vivo.” One function of the three dimensional environment is to direct cell behavior such as migration, proliferation, differentiation, maintenance of the phenotypes and apoptosis by facilitating sensing, and responding to the environment via cell-matrix and cell-cell communications. Therefore, a material having proper porosity, large surface area, and well inter-connected pores is desirable for culturing cells. In particular, to achieve efficient cell culturing that is comparable to in vivo cell growth, it is desirable that the material permit the permeation of cells through the entire material.

Substrates for growing cells generally have a solid, non-porous base substrate that provides a support for a membrane. Because the base substrate is non-porous, cell permeation is not possible. This ultimately limits the ability of the substrate to mimic a three-dimensional in vivo matrix and, subsequently, its use as a scaffold for growing and harvesting cells. Described herein are substrates that facilitate the immobilization and subsequent growth of cells and tissues. The substrates described herein more closely resemble in vivo three-dimensional matrices, which have numerous applications.

### SUMMARY

In accordance with the purposes of the disclosed materials, compounds, compositions, articles, devices, and methods, as embodied and broadly described herein are substrates for immobilizing cells and tissues and methods of use thereof. Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIG. 1 shows a nanofiber mat composed of PLGA nanofibers having a diameter less than 2 μm.

FIG. 2 is a SEM picture showing the front-side of a PLGA/Nylon membrane.

FIG. 3 is a SEM picture showing the backside of a PLGA/Nylon membrane.

### DETAILED DESCRIPTION

The materials, compounds, compositions, articles, devices, and methods described herein may be understood more readily by reference to the following detailed description of specific aspects of the disclosed subject matter and the Examples included therein and to the Figures.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the layer” includes mixtures of two or more such layers, and the like. “Optional” or “optionally” means that the subsequently described event or circumstance can or cannot occur, and that the description includes instances where the event or circumstance occurs and instances where it does not.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers or prepared by methods known to those skilled in the art.

Also, disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a composition is disclosed and a number of modifications that can be made to a number of components of the composition are discussed, each and every combination and permutation that are possible are specifically contemplated unless specifically indicated to the contrary. Thus, if a class of components A, B, and C are disclosed as well as a class of components D, E, and F and an example of a composition A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

Reference will now be made in detail to specific aspects of the disclosed materials, compounds, compositions, articles, and methods, examples of which are illustrated in the accompanying Examples and Figures.

Each component of the substrates and the applications of the substrates are described below.

### I. Substrates For Cell/Tissue Immobilization

Described herein are substrates for immobilizing cells or tissues. Upon immobilization of the cells or tissues, numerous applications are contemplated. These applications will be described below.

In one aspect, described herein is a substrate for immobilizing cells or tissue comprising:
a. a network of nanofibers, and
b. a base substrate comprising a non-woven or woven porous substrate, wherein the base substrate comprises a first outer surface, wherein the network of nanofibers is adjacent to the first outer surface of the base substrate.

Each component of the substrates described herein is discussed below.

a. Network of Nanofibers

The term “nanofiber” as used herein means a polymer fine fiber comprising a diameter of about 1000 nanometers or less. The term “network” as used herein means a random or oriented distribution of nanofibers in space that is controlled to form an interconnecting net with spacing between fibers selected to promote growth and culture stability. The network has small spaces between the fibers comprising the network forming pores or channels in the network. The size of the pores or channels can vary depending upon the cell or tissue to be immobilized. In one aspect, the pore size of the nanofiber network is greater than 0.2 microns. In another aspect, the pore size is less than 1 micron. In a further aspect, the pore size is from 0.2 microns to 300 microns. The network can comprise a single layer of nanofibers, a single layer formed by a continuous nanofiber, multiple layers of nanofibers, multiple layers formed by a continuous nanofiber, or mat. The network may be unwoven or net. Physical properties of the network include, but are not limited to, texture, rugosity, adhesivity, porosity, solidity, elasticity, geometry, interconnectivity, surface to volume ratio, fiber diameter, fiber solubility/insolubility, hydrophilicity/hydrophobicity, fibril density, and fiber orientation.

The network of nanofibers comprises one or more polymers. The selection of polymer(s) can vary depending upon the application of the substrate. In various aspects, the polymer can be water soluble or insoluble. In other aspects, the polymer is biodegradable, biocompatible, and/or non-cytotxic. In the case two or more polymers are used to produce the nanofiber network, the polymers can be blended prior to nanofiber formation or, in the alternative, nanofibers can be independently formed from each polymer followed by mixing each fiber.

The polymers can be derived from natural or synthetic fibers. Examples of natural fibers include, but are not limited to, a protein, a polysaccharide, a cellulose derivative, or a mixture thereof. Examples of synthetic fibers include, but are not limited to, a polyester, a polyamide, or a mixture thereof.

In other aspects, polymer materials that can be used to produce nanofibers include both addition polymer and condensation polymer materials such as a polyolefin, cyclic polyolefin, polyacetal, polyamide, polyester, polycarbonate, cellulose ether and ester, polyalkylene sulfide, polyarylene oxide, polyalkylene oxide, copolymers and block copolymers of alkylene oxide, polyvinylcarbazole, polysulfone, modified polysulfone polymers and mixtures thereof. Preferred materials that fall within these generic classes include polyethylene, poly(epsilon-caprolactone), a polylactide, a polyglycolide, a polylactide-co-glycolide, polypropylene, polysiloxane, poly(vinylchloride), polyvinylpyrrolidone, polyvinyl acetate, polymethylmethacrylate (and other (meth)acrylic resins), poly (meth)acrylamide, polystyrene, and copolymers thereof (including ABA type block copolymers), poly(vinylidene fluoride), poly(vinylidene chloride), polyvinyl alcohol in various degrees of hydrolysis (87% to 99.5%) in crosslinked and non-crosslinked forms.

In one aspect, the polymer is a polyester. Aliphatic polyesters such as poly(epsilon-caprolactone), poly(lactate), poly(glycolate), and their copolymers are biodegradable and biocompatible.

In another aspect, the polymer is a polyamide. One class of polyamide condensation polymers is nylon materials. The term “nylon” is a generic name for all long chain synthetic polyamides. Typically, nylon nomenclature includes a series of numbers such as in nylon-6,6, which indicates that the starting materials are a C₆ diamine and a C₆ diacid (the first digit indicating a C₆ diamine and the second digit indicating a C₆ dicarboxylic acid compound). Another nylon can be made by the polycondensation of epsilon caprolactam in the presence of a small amount of water. This reaction forms a nylon-6 (made from a cyclic lactam—also known as epsilonaminocaproic acid) that is a linear polyamide. Further, nylon copolymers are also contemplated. Copolymers can be made by combining various diamine compounds, various diacid compounds, and various cyclic lactam structures in a reaction mixture and then forming the nylon with randomly positioned monomeric materials in a polyamide structure. For example, a nylon 6,6-6,10 material is a nylon manufactured from hexamethylene diamine and a C₆ and a C₁₀ blend of diacids. A nylon 6-6,6-6,10 is a nylon manufactured by copolymerization of epsilon aminocaproic acid, hexamethylene diamine and a blend of a C₆ and a C₁₀ diacid material.

Block copolymers are also useful with respect to nanofiber formation. Examples of block copolymers useful herein include, but are not limited to, Kraton® type of AB and ABA block polymers including styrene/butadiene and styrene/hydrogenated butadiene(ethylene propylene), Pebax® type of epsilon-caprolactam/ethylene oxide, Sympatex® polyester/ethylene oxide and polyurethanes of ethylene oxide and isocyanates.

In certain aspects, two or more polymer materials can be blended for beneficial properties. Polymer blends can improve physical properties by changing polymer attributes such as improving polymer chain flexibility or chain mobility, increasing overall molecular weight and providing reinforcement through the formation of networks of polymeric materials.

b. Preparation of Nanofiber Network

The nanofibers can be fabricated using techniques known in the art. Polymer selection and/or the process by which the nanofibers are fabricated and/or directed and oriented onto a substrate allow for specific selection and manipulation of physical properties of the nanofiber network. Physical properties of the nanofiber network, including fiber size, fiber diameter, fiber spacing, matrix density, fiber texture and elasticity, can be important considerations for organizing the cytoskeletal networks in cells and the exposure of cell signaling motifs in extracellular matrix proteins. Physical properties of the nanofiber network that can be engineered to desired parameters include, but are not limited to, texture, rugosity, adhesivity, porosity, solidity, elasticity, geometry, interconnectivity, surface to volume ratio, fiber size, fiber diameter, fiber solubility/insolubility, hydrophilicity/hydrophobicity, and fibril density.

One or more of the physical properties of the nanofiber network can be varied and/or modified to create a specifically defined environment for cell immobilization. For example, porosity of the nanofiber network can be engineered to enhance diffusion of ions, metabolites, and/or bioactive molecules and/or allow cells to penetrate and permeate the nanofiber network to grow in an environment that promotes multipoint attachments between the cells and the nanofiber network. Interconnectivity of the nanofiber network can be engineered to facilitate cell-cell contacts. Elasticity of the nanofiber network can be increased or decreased by adding a bioactive molecule to the polymer solution from which the nanofibers are fabricated. It is also possible to produce nanofibers that are hollow or have a core with a sheath.

Texture and rugosity of the nanofiber network can be engineered to promote attachment of cells. For example, homogeneous or heterogeneous nanofibers can be selected to optimize growth or differentiation activity of the cells. In one aspect, the nanofiber network comprises multiple nanofibers having different diameters and/or multiple nanofibers fabricated from different polymers. In other aspects, the solubility or insolubility of the nanofibers of the nanofiber network can be engineered to control the release of bioactive molecules that can be incorporated into the nanofiber network. For example, the rate of release of bioactive molecules is determined by the rate of biodegradation or biodissolution of the nanofibers of the nanofiber network. In other aspects, the hydrophobicity and hydrophilicity of the nanofiber network can be engineered to promote specific cell spacing.

In one aspect, the nanofiber network can be produced by charging techniques such as, for example, corona charging and tribocharging. In another aspect, the nanofibers can be prepared by electrospinning techniques. The electrospinning process uses an electric field to control the formation and deposition of polymers. A polymer solution is injected with an electrical potential. The electrical potential creates a charge imbalance that leads to the ejection of a polymer solution stream from the tip of an emitter such as a needle. The polymer jet within the electric field is directed toward a grounded substrate, during which time the solvent evaporates and fibers are formed. The resulting single continuous filament collects as a nonwoven fabric on the base substrate.

The nanofiber networks can be produced having random or directed orientations. Random fibers can be assembled into layered surfaces. In one aspect, the nanofibers comprise a random distribution of fine fibers that can be bonded to form an interlocking network. The nanofiber interlocking networks have relatively small spaces between the fibers. Such spaces form pores or channels in the nanofiber network allowing for diffusion of ions, metabolites, proteins, and/or bioactive molecules as well as cells to penetrate and permeate the network and grow in an environment that promotes multipoint attachments between cells and the nanofibers.

In other aspects, nanofiber networks can be electrospun in an oriented manner. Such oriented electrospinning allows for the fabrication of a nanofiber network comprising a single layer of nanofibers or a single layer formed by a continuous nanofiber, wherein the network has a height of the diameter of a single nanofiber. Physical properties including porosity, solidity, fibril density, texture, rugosity, and fiber orientation of the single layer network can be selected by controlling the direction and/or orientation of the nanofiber onto the substrate during the electrospinning process. Preferably the pore size allows cells to penetrate and/or migrate through a single layer or multi-layer nanofiber network.

The layering of individual single layer nanofiber networks can form channels, which allow diffusion of ions, metabolites, proteins, and/or bioactive molecules as well as permit cells to penetrate the nanofiber network and grow in an environment that promotes multipoint attachments between the cells and the nanofiber network.

The morphology and physical properties of the nanofiber network can vary depending upon, among other things, the selection of the polymer, the conformation of the polymer chain, and the solvent used. In one aspect, phase separation techniques can be used to fabricate the nanofiber network. The phase separation process generally involves polymer dissolution, phase separation and gelation, solvent extraction from the gel with water, freezing, and then freeze drying under a vacuum. By varying the ratio of polymers and the solvents, it is possible to control the topography of the nanofibers.

C. Base Substrate

The term “base substrate” as used herein means a surface on which the network of nanofibers can be deposited. The base substrate offers structural support for the deposited network of nanofibers. In one aspect, the base substrate comprises a non-woven or woven porous substrate. Techniques for producing woven and non-woven porous materials are known in the art. In one aspect, the base substrate comprises a mat produced from woven or non-woven materials.

The base substrate is porous. Depending upon the porosity of the nanofiber network, the base substrate can have pores that are greater or smaller in diameter to the pores present in the nanofiber network. It is contemplated that cells can penetrate and be retained by the base substrate and/or the network of nanofibers. The size of the pores in the base substrate can vary depending upon the cell or tissue to be immobilized. In one aspect, the pore size is greater than 0.2 microns. In another aspect, the pore size is less than 1 micron. In a further aspect, the pore size is from 0.2 microns to 300 microns.

In another aspect, the base substrate is composed of one or more polymers. Examples of such polymers include, but are not limited to, a polyolefin, cyclic polyolefin, polyacetal, polyamide, polyester, polycarbonate, cellulose ether and ester, polyalkylene sulfide, polyarylene oxide, polyalkylene oxide, copolymers and block copolymers of alkylene oxide, polyvinylcarbazole, polysulfone, modified polysulfone polymers and mixtures thereof. Preferred materials that fall within these generic classes include polyethylene, poly(epsilon-caprolactone), a polylactide, a polyglycolide, a polylactide-co-glycolide, polypropylene, polysiloxane, poly(vinylchloride), polyvinylpyrrolidone, polyvinyl acetate, polymethylmethacrylate (and other (meth)acrylic resins), poly (meth)acrylamide, polystyrene, and copolymers thereof (including ABA type block copolymers), poly(vinylidene fluoride), poly(vinylidene chloride), polyvinyl alcohol in various degrees of hydrolysis (87% to 99.5%) in crosslinked and non-crosslinked forms or mixtures thereof. It is contemplated that the base substrate can be composed of layers of different polymers or composed of a blend of two or more polymers. Any of the polymers described above can be woven or non-woven to produce the base substrate. For example, the base substrate can be composed of Nylon fibers woven into a mat.

d. Bioactive Molecules

The nanofiber network and/or the base substrate can comprise one or more bioactive molecules. In one aspect, the network of nanofibers or base substrate comprises one or more compounds for enhancing cell growth. In another aspect, the nanofiber or base substrate further comprises a compound that promotes attachment of a cell or tissue to the nanofiber or substrate.

Bioactive molecules include human or veterinary therapeutics, nutraceuticals, vitamins, salts, electrolytes, amino acids, peptides, polypeptides, proteins, carbohydrates, lipids, polysaccharides, nucleic acids, nucleotides, polynucelotides, glycoproteins, lipoproteins, glycolipids, glycosaminoglycans, proteoglycans, growth factors, differentiation factors, hormones, neurotransmitters, pheromones, chalones, prostaglandins, immunoglobulins, monokines and other cytokines, humectants, minerals, electrically and magnetically reactive materials, light sensitive materials, anti-oxidants, molecules that may be metabolized as a source of cellular energy, antigens, and any molecules that can cause a cellular or physiological response. Any combination of molecules can be used, as well as agonists or antagonists of these molecules. Glycoaminoglycans include glycoproteins, proteoglycans, and hyaluronan. Polysaccharides include cellulose, starch, alginic acid, chytosan, or hyaluronan. Cytokines include, but are not limited to, cardiotrophin, stromal cell derived factor, macrophage derived chemokine (MDC), melanoma growth stimulatory activity (MGSA), macrophage inflammatory proteins 1 alpha (MIP-1 alpha), 2, 3 alpha, 3 beta, 4 and 5, interleukin (IL) 1, IL-2, IL-3, IL-4, IL-5, IL-6, IL-7, IL-8, IL-9, IL-10, IL-11, IL-12, IL-13, TNF-alpha, and TNF-beta. Immunoglobulins useful in the present invention include, but are not limited to, IgG, IgA, IgM, IgD, IgE, and mixtures thereof. Amino acids, peptides, polypeptides, and proteins can include any type of such molecules of any size and complexity as well as combinations of such molecules. Examples include, but are not limited to, structural proteins, enzymes, and peptide hormones.

The term bioactive molecule also includes fibrous proteins, adhesion proteins, adhesive compounds, deadhesive compounds, and targeting compounds. Fibrous proteins include collagen arid elastin. Adhesion/deadhesion compounds include fibronectin, laminin, thrombospondin and tenascin C. Adhesive proteins include actin, fibrin, fibrinogen, fibronectin, vitronectin, laminin, cadherins, selectins, intracellular adhesion molecules 1, 2, and 3, and cell-matrix adhesion receptors including but not limited to integrins such as α₅β₁, α₆β₁, α₇β₁, α₄β₂, α₂β₃, and α₆β₄.

The term bioactive molecule also includes leptin, leukemia inhibitory factor (LIF), RGD peptide, tumor necrosis factor alpha and beta, endostatin, angiostatin, thrombospondin, osteogenic protein-1, bone morphogenic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12, 13, 15, 16, 17 and 18.

The term “growth factor” as used herein means a bioactive molecule that promotes the proliferation of a cell or tissue. Growth factors useful in the present invention include, but are not limited to, transforming growth factor-alpha. (TGF-alpha), transforming growth factor-beta. (TGF-beta), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, and beta3, skeletal growth factor, bone matrix derived growth factors, and bone derived growth factors and mixtures thereof. Some growth factors can also promote differentiation of a cell or tissue. TGF, for example, can promote growth and/or differentiation of a cell or tissue. Some preferred growth factors include VEGF, NGFs, PDGF-AA, PDGF-BB, PDGF-AB, FGFb, FGFa, and BGF.

The term “differentiation factor” as used herein means a bioactive molecule that promotes the differentiation of cells or tissues. The term includes, but is not limited to, neurotrophin, colony stimulating factor (CSF), or transforming growth factor. CSF includes granulocyte-CSF, macrophage-CSF, granulocyte-macrophage-CSF, erythropoietin, and IL-3. Some differentiation factors may also promote the growth of a cell or tissue. TGF and IL-3, for example, can promote differentiation and/or growth of cells.

The term “adhesive compound” as used herein means a bioactive molecule that promotes attachment of a cell or tissue to a fiber surface comprising the adhesive compound. Examples of adhesive compounds include, but are not limited to, fibronectin, vitronectin, and laminin.

The term “deadhesive compound” as used herein means a bioactive molecule that promotes the detachment of a cell or tissue from a fiber comprising the deadhesive compound. Examples of deadhesive compounds include, but are not limited to, thrombospondin and tenascin C.

The term “targeting compound” as used herein means a bioactive molecule that functions as a signaling molecule inducing recruitment and/or attachment of cells or tissues to a fiber comprising the targeting compound. Examples of targeting compounds and their cognate receptors include attachment peptides including RGD peptide derived from fibronectin and integrins, growth factors including EGF and EGF receptor, and hormones including insulin and insulin receptor.

The bioactive molecules can be incorporated into the nanofiber network or the base substrate during fabrication of the network or substrate or can be attached to a surface of the network or substrate via a functional group. In certain aspects, one or more functional groups can be incorporated on the outside surface of the nanofibers or base substrate. These functionalized surfaces can bind a peptide, polypeptide, lipid, carbohydrate, polysaccharide, amino acid, nucleotide, nucleic acid, polynucleotide, or other bioactive molecules to the surface of the nanofiber or base substrate. In one aspect, the functional groups are deposited on the outside surface of the nanofiber or base substrate by plasma deposition. Plasma deposition creates local plasmas at the surface of the nanofiber or base substrate. The treated surface is then reacted with gaseous molecules, such as for example, allylamine and/or allyl alcohol, in a reaction chamber. In another aspect, the functional groups are introduced onto the surface of the nanofibers during the electrospinning process. For example, dodecyl amine, dodecyl aldehyde, dodecyl thiol, or dodecyl alcohol can be added to the polymer solution. The polymer solution is then electrospun into nanofibers in which a portion of the added amines, aldehydes, sulphydryl, or alcohol moieties, respectively, are exposed on the outside surface of the nanofibers.

### II. Preparation of Substrates For Cell/Tissue Immobilization

The nanofiber network can be deposited on the base substrate using techniques known in the art. In one aspect, the nanofiber network can be produced and deposited on the base substrate by charging techniques such as, for example, corona charging and tribocharging. Alternatively, the nanofiber network can be electrospun onto the base substrate such that the nanofiber network is adjacent to the base substrate. In other aspects, a preformed nanofiber network can be attached to the base substrate with the use of an adhesive.

The term “adjacent” as used herein includes the intimate contact between the nanofiber network and the surface of the base substrate. The term “adjacent” also includes one or more layers interposed between the nanofiber network and the base substrate. For example, an adhesion protein can be deposited on the outer surface of the base substrate prior to depositing the nanofiber network on the base substrate. In one aspect, cells or tissue are not interposed between the nanofiber network and the base substrate. As described above, electrospinning can be used to produce nanofibers with different properties and orientations as desired. In general, although not prohibited, the other exposed surface of the base substrate does not have any components adjacent to the other exposed surface. Upon deposition of the nanofibers on the base substrate, the nanofibers are evenly distributed on the base substrate at a uniform thickness.

It is also contemplated that two or more nanofiber networks can be layered on the base substrate. For example, different nano- and/or micro-environments that promote cellular activity of a particular cell or tissue can be constructed by layering different nanofiber networks that have selected physical and/or chemical properties. The physical and/or chemical properties can be engineered into the individual nanofiber networks as described above. The layering of individual nanofiber networks can form channels that allow diffusion of ions, metabolites, proteins, and/or bioactive molecules as well as permit cells to penetrate the substrate and grow in an environment that promotes multipoint attachments between the cells and the nanofiber network.

### III. Kits

In another aspect, described herein is a kit comprising a network of nanofibers and a base substrate. Any of the nanofiber networks and base substrates described above can be used herein. In one aspect, one or more pre-manufactured nanofiber networks can be individually wrapped and sterilized. After removal from the packaging, one or more nanofiber networks can be assembled manually or mechanically on the base substrate. In the case of multiple nanofiber networks, each nanofiber network can be applied to the base substrate layer by layer to form a multi-layered assembly.

### IV. Applications

The substrates described herein are used to immobilize cells or tissues. The term “immobilization” as used herein is the ability of the substrate to retain the cell or tissue. Immobilization can range from completely retaining the cell or tissue such that the cell or tissue is locked in position within the nanofiber network or base substrate to a situation where the cell or tissue can freely permeate the nanofiber network or base substrate. The incorporation of bioactive molecules into the nanofiber network or base substrate can determine the degree of immobilization of the cell or tissue on the substrate.

The substrates described herein can be used in a number of applications, which are described below. It is contemplated that the substrates can be used in many known applications employing nanofibers including, but not limited to, filter applications, pharmaceutical applications, cell culture, tissue culture, and tissue engineering. It is contemplated one or more cell types can be deposited on the substrate. The cells can be deposited on the substrate using techniques known in the art.

In one aspect, described herein is a method for growing a plurality of cells, comprising (a) depositing a parent set of cells on a substrate described herein, and (b) culturing the substrate with the deposited cells to promote the growth of the cells.

In another aspect, described herein is a method for differentiating cells, comprising (a) depositing a parent set of cells on a substrate described herein, and (b) culturing the assembly to promote differentiation of the cells.

Many types of cells can be immobilized on the substrate including, but not limited to, stem cells, committed stem cells, differentiated cells, and tumor cells. Examples of stem cells include, but are not limited to, embryonic stem cells, bone marrow stem cells and umbilical cord stem cells. Other examples of cells used in various embodiments include, but are not limited to, osteoblasts, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, and neurons.

Cells useful herein can be cultured in vitro, derived from a natural source, genetically engineered, or produced by any other means. Any natural source of prokaryotic or eukaryotic cells can be used.

Atypical or abnormal cells such as tumor cells can also be used herein. Tumor cells cultured on substrates described herein can provide more accurate representations of the native tumor environment in the body for the assessment of drug treatments. Growth of tumor cells on the substrates described herein can facilitate characterization of biochemical pathways and activities of the tumor, including gene expression, receptor expression, and polypeptide production, in an in vivo-like environment allowing for the development of drugs that specifically target the tumor.

Cells that have been genetically engineered can also be used herein. The engineering involves programming the cell to express one or more genes, repressing the expression of one or more genes, or both. Genetic engineering can involve, for example, adding or removing genetic material to or from a cell, altering existing genetic material, or both. Embodiments in which cells are transfected or otherwise engineered to express a gene can use transiently or permanently transfected genes, or both. Gene sequences may be full or partial length, cloned or naturally occurring.

By varying and/or modifying selected physical and/or chemical properties of the substrate, the substrate can be engineered to promote cellular growth of a particular cell or tissue. The physical properties and/or characteristics of the substrate including, but not limited to, texture, rugosity, adhesivity, porosity, elasticity, solidity, geometry, and fibril density can be varied and/or modified to promote a desired cellular activity, including growth and/or differentiation. Specific nano- and/or micro-environments can be engineered within the substrate. For example, the porosity and fibril density of the substrate can be varied and/or modified to allow a cell to penetrate the substrate and grow in a three dimensional environment. Any of the bioactive molecules described herein can be engineered into the substrate either isotropically or as gradients to promote desired cellular activity, including cell adhesion, growth, and/or differentiation. The physical and/or chemical properties of the substrate, including growth and differentiation factors, on which such cells are grown can be engineered to mimic the native in vivo nano- or micro-environments.

With designed patterns, the spatial organization of the cells in two and three dimensions can be obtained. By creating specific patterns of surface chemistry, cell behavior can be confined within physical or chemical ultrastructures, which can be used to control cellular activity such as cell growth and/or proliferation.

In another aspect, described herein is method for growing tissue, comprising (a) depositing a parent set of cells that are a precursor to the tissue on a substrate described herein, and (b) culturing the substrate with the deposited cells to promote the growth of the tissue. It is also contemplated that viable cells can be deposited on the substrates described herein and cultured under conditions that promote tissue growth. Tissue grown (i.e., engineering) from any of the cells described above is contemplated with the substrates described herein. The supports described herein can support many different kinds of precursor cells, and the substrates can guide the development of new tissue. The production of tissues has numerous applications in wound healing. Depending upon the selection of materials used to produce the nanofibers and base substrate, tissue growth can be performed in vivo or ex vivo.

In certain instances, it is desirable to remove the cells or tissue from the substrate. For example, it would be desirable to harvest stem cells that have been growing on the substrates described herein. Invasive techniques known in the art for removing cells include, but are not limited to, mechanical scraping, sonication, chemical/enzymatic treatment, or a combination thereof. Other techniques involve adjusting the pH or temperature or the addition of ions to release attached cells.

In another aspect, described herein are methods for determining an interaction between a known cell line and a drug, comprising (a) depositing the known cell line on a substrate described herein; (b) contacting the deposited cells with the drug; and (c) identifying a response produced by the deposited cells upon contact with the drug.

With a known cell line immobilized on the substrates described herein, it is possible to screen the activity of several drugs when the drug interacts with the immobilized cells. Depending upon the cells and drugs to be tested, the cell-drug interaction can be detected and measured using a variety of techniques. For example, the cell may metabolize the drug to produce metabolites that can be readily detected. Alternatively, the drug can induce the cells to produce proteins or other biomolecules. The substrates described herein provide an environment for the cells to more closely mimic the in vivo nature of the cells in an ex vivo environment. The substrates can be used in high throughput applications for analyzing drug/cell interactions. High throughput applications utilize multiwell tissue culture chambers with densities up to about 1536 wells per plate. Thus, increasing the population of cells per well would serve to increase the measured signals.

In another aspect, described herein are methods for separating a compound present in a solution, comprising (a) contacting the solution with a substrate described herein, wherein the compound is immobilized on the substrate; and (b) removing the immobilized compound from the substrate. The nanofiber network and/or base substrate can be modified to immobilize particular biomolecules in solution. In general, a solution composed of one or more biomolecules is contacted with the substrate, at which time the biomolecule is immobilized on the substrate. The bound biomolecule can then be released from the substrate with a solvent. The substrate can be modified so that the substrate forms a covalent or non-covalent (e.g., ionic, electrostatic dipole-dipole, Van Der Waals interactions) bond with the biomolecule. In another aspect, cells can be purified. For example, by measuring the electric properties of a single individual cell immobilized on the substrate, it is possible to sort/purify a population of cells by their different intrinsic electric properties. This application can be of particular interest in stem cells, where it is desirable to harvest large quantities of pure stem cells.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

### Example 1

### Preparation of A PLGA Nanofiber Mat Deposited Onto A Nylon Woven Substrate

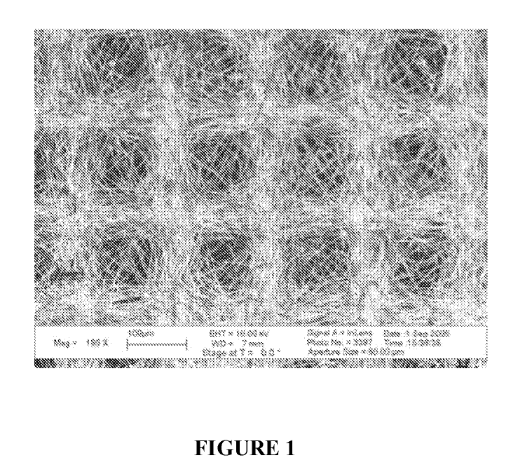

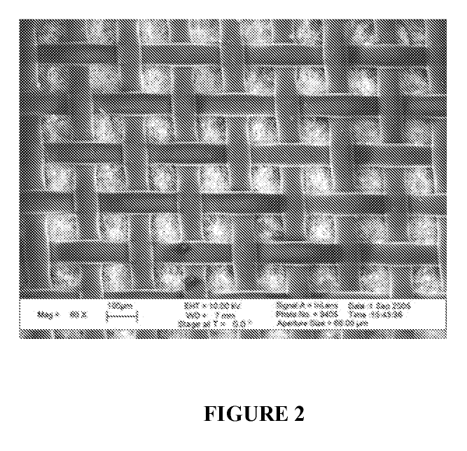

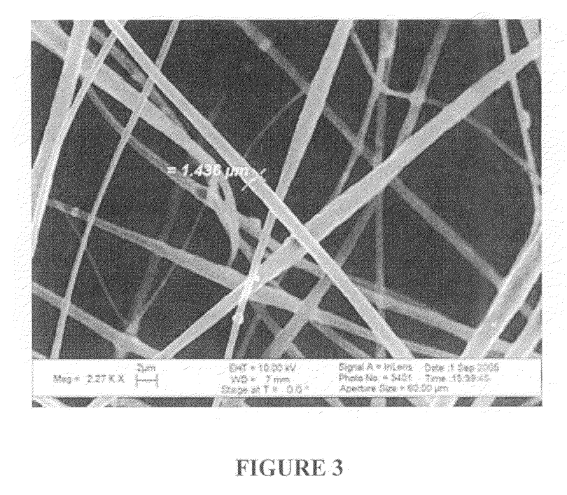

A solution of 20% PLGA (85-15) in 80% THF/20% DMF was electrospun. The applied electric field was about 1 kV/cm and the flow rate was 0.03 ml/min. The spinneret was connected to a source of +15,000 Volts. The collector was a Nylon woven membrane deposited on the top of an aluminum grounded electrode. After a few minutes of deposition, a nanofiber mat composed of fibers having a diameter less than 2 micron was obtained (FIG. 1). FIGS. 2 and 3 are SEM pictures that show the structure of the membrane from the front side and from the backside, respectively.

Throughout this application, various publications are referenced. The disclosures of these publications in their entireties are hereby incorporated by reference into this application in order to more fully describe the compounds, compositions and methods described herein.

Various modifications and variations can be made to the materials, methods, and articles described herein. Other aspects of the materials, methods, and articles described herein will be apparent from consideration of the specification and practice of the materials, methods, and articles disclosed herein. It is intended that the specification and examples be considered as exemplary.

What is claimed is:

1. A substrate for immobilizing cells or tissue comprising:
a. a network of nanofibers, and
b. a base substrate comprising a non-woven or woven porous substrate, wherein the base substrate comprises a first outer surface, wherein the network of nanofibers is adjacent to the first outer surface of the base substrate.

2. The substrate of claim 1, wherein the network of nanofibers has a pore size less than the diameter of the cell or tissue.

3. The substrate of claim 1, wherein the base substrate has a pore size less than the diameter of the cell or tissue.

4. The substrate of claim 1, wherein the network of fibers has a pore size greater than 0.2 μm.

5. The substrate of claim 1, wherein the network of fibers has a pore size less than 1 μm.

6. The substrate of claim 1, wherein the base substrate has a pore size greater than 0.2 μm.

7. The substrate of claim 1, wherein the network of nanofibers comprises electrospun natural or synthetic fibers.

8. The substrate of claim 1, wherein the network of nanofibers comprises a polyolefin, cyclic polyolefin, polyacetal, polyamide, polyester, polycarbonate, cellulose ether and ester, polyalkylene sulfide, polyarylene oxide, polyalkylene oxide, copolymers and block copolymers of alkylene oxide, polyvinylcarbazole, polysulfone, a modified polysulfone polymer, or a mixture thereof.

9. The substrate of claim 1, wherein the base substrate comprises a polyolefin, cyclic polyolefin, polyacetal, polyamide, polyester, polycarbonate, cellulose ether and ester, polyalkylene sulfide, polyarylene oxide, polyalkylene oxide, copolymers and block copolymers of alkylene oxide, polyvinylcarbazole, polysulfone, a modified polysulfone polymer, or a mixture thereof.

10. The substrate of claim 1, wherein the base substrate comprises a polyamide.

11. The substrate of claim 1, wherein the network of nanofibers comprises a polylactide-co-glycolide and the base substrate comprises a woven substrate comprising polyamide.

12. The substrate of claim 1, wherein the network of nanofibers, the base substrate, or a combination thereof further comprises one or more bioactive molecules.

13. A kit comprising one or more networks of nanofibers and base substrate comprising a non-woven porous substrate or a woven porous substrate.

14. A method for growing a plurality of cells, comprising (a) depositing a parent set of cells on the substrate of claim 1, and (b) culturing the substrate with the deposited cells to promote the growth of the cells.

15. A method for differentiating cells, comprising (a) depositing a parent set of cells on the substrate of claim 1, and (b) culturing the substrate to promote differentiation of the cells.

16. A method for determining an interaction between a known cell line and a drug, comprising (a) depositing the known cell line on the substrate of claim 1; (b) contacting the deposited cells with the drug; and (c) identifying a response produced by the deposited cells upon contact with the drug.

17. A method for separating a compound present in a solution, comprising (a) contacting the substrate of claim 1 with the solution, wherein the compound is immobilized on the substrate; and (b) removing the immobilized compound from the substrate.

18. A method for growing tissue, comprising (a) depositing a parent set of cells that are a precursor to the tissue on the substrate of claim 1, and (b) culturing the substrate with the deposited cells to promote the growth of the tissue.
